(19) United States
(12) Patent Application Publication
(10) Pub. No.: US 2018/0028100 A1
(43) Pub. Date: Feb. 1, 2018

(54) SYSTEM AND NON-INVASIVE METHOD FOR EXAMINING AT LEAST PARTS OF BLOOD FRACTIONS, AND USE OF THE SYSTEM

(71) Applicant: SIEMENS AKTIENGESELLSCHAFT, München (DE)

(72) Inventors: Sebastian Martius, Forchheim (DE); Benjamin Sewiolo, Obermichelbach (DE); Andreas Ziroff, München (DE)

(21) Appl. No.: 15/550,334

(22) PCT Filed: Feb. 11, 2015
(86) PCT No.: PCT/EP2015/052878

§ 371 Date: Aug. 10, 2017

(51) Int. Cl. A61B 5/145; A61B 5/00; A61B 5/05; G01R 33/465; A61B 5/1495; A61B 5/01; G01R 33/3815; G01R 33/383; A61B 5/055; A61B 8/08

(52) U.S. Cl. CPC A61B 5/14532; A61B 5/055; A61B 5/4866; A61B 5/0035; A61B 5/0075; A61B 5/0507; A61B 8/488; A61B 5/1495; A61B 5/6826; A61B 5/01; G01R 33/3815; G01R 33/383; G01R 33/465; G01R 33/281

(57) ABSTRACT

The invention relates to a system and a non-invasive method for examining at least parts of the blood fractions, designed for examination of the blood fractions on the basis of at least one magnetic resonance spectroscopy of a human body part, in particular of the human finger. The invention also relates to the use of said system.

The present patent document is a §371 nationalization of PCT Application Serial Number PCT/EP2015/052878, filed Feb. 11, 2015, designating the United States, which is hereby incorporated by reference.

### BACKGROUND

The present embodiments relate to noninvasively examining at least parts of blood constituents.

The examination of the blood (e.g., the blood sugar content) is a process that is known from the field of medical examinations. Blood taken either by a cannula or by pricking the finger is examined.

Blood taken by pricking the finger is a daily process for many diabetics. Patients with type-1 diabetes, for example, frequently measure blood sugar values. Currently, there is no reliable noninvasive method to make this measurement more bearable for the patient.

Very different approaches, such as optical methods, gas analysis (e.g., of respiration), biosensors, microwave-based and millimeter-wave-based methods, etc. have been examined as possible noninvasive methods. To date, no reliable noninvasive method has been developed.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, noninvasive examination of blood that has a higher reliability and a moderate calibration outlay compared to the prior art is provided.

The present embodiments will be explained in more detail below. The arrangement according to one or more of the present embodiments for noninvasively examining at least parts of the blood constituents is configured to examine the blood constituents based on at least a magnetic resonance spectroscopy of a part of the human body (e.g., of a human finger).

A miniaturization that makes the examination accessible to a broad field of application is rendered possible by focusing the examination on a part of the body. In conjunction with dispensing with an intervention in the body, which is rendered possible by the focused use of at least magnetic resonance spectroscopy, this examination becomes more comfortable for patients.

The arrangement has degrees of freedom that facilitate a configuration or implementation that is matched to the requirements of use. Further, the risks of infection are significantly minimized therewith, both for medical personnel and for the person to be examined. Devices that are configured in accordance with one or more of the present embodiments are easy to sterilize.

In accordance with a development, the arrangement is configured for the examination by the capture of at least one blood substance based on at least magnetic resonance spectroscopy.

Focusing directly on blood substances permits a further minimization of the arrangement (e.g., because it is possible, via the blood flow, to capture the concentration of the blood substance/substances ascertained over time and not over examined area, as would be the case in other tissue parts). Therefore, the area of the examined position or of the examined body part may be selected to be very small.

A direct capture is suitable for capturing current blood sugar values, as is provided in a development of the arrangement. In the development, the arrangement is configured for the examination by the capture of at least the blood substance glucose, based on at least magnetic resonance spectroscopy.

Alternatively, or in addition, one or more of the present embodiments may be developed such that the arrangement is configured for the examination by the capture of at least one blood substance, at least via a marker substance that correlates with the blood substance, based on at least magnetic resonance spectroscopy.

As an alternative, this development is advantageous if the examination of the blood substance by direct capture of the substance appears unsuitable or impossible. However, if it is possible in principle and implemented, this development may be effectuated to complement the direct capture (e.g., in order to increase the reliability of the value of the ascertained concentration of the substance in the blood). This may also be used for calibration purposes.

For the purposes of examining the concentration of the blood sugar, the arrangement may be configured for the examination by the capture of the blood substance glucose via at least one marker substance that correlates with the blood substance glucose (e.g., one of the known glucose transporters “GLUT”, such as “GLUT-1” . . . “GLUT-12”, insulin receptor “INSR” or the like), based on at least magnetic resonance spectroscopy. For marker substances (e.g., for sugar), the present research in relation to these substances yields the expectation of reliable values when ascertaining the concentration on the basis thereof.

As an alternative or in addition thereto, the arrangement may be configured for the examination by the capture of the blood sugar metabolism, via at least a correlating marker substance (e.g., based on the hydrogen atom nuclei “¹H-MRS” and/or the carbon atom nuclei “¹³C-MRS”), based on at least magnetic resonance spectroscopy. The arrangement is configured with device for evaluating the capture. The device is configured such that the blood sugar concentration is derived from the spectrum that belongs to the marker substance.

The present research in relation to these substances, which are known from the metabolism of the blood sugar, likewise yields the expectation of the suitability for reliable values. This also renders possible a calibration or an extension of the examination based on the direct capture or other correlating substances, and hence also degrees of freedom for a further improvement in the reliability.

In one embodiment, for the examination by the capture based on magnetic resonance spectroscopy, the arrangement includes a device for carrying out magnetic resonance imaging (e.g., a magnetic resonance imaging device), with a device for carrying out a Doppler measurement, a device for carrying out an infrared measurement, a device for carrying out a microwave-based measurement, and/or with sensors such as, for example, temperature sensors. The magnetic resonance imaging device is configured in a manner functionally linked therewith such that the capture is carried out depending on a measurement that is effectuated by the sensors.

As a result of this, use is made of further noninvasive measurement methods in order to adapt at least the examination that is carried out by the magnetic resonance spectroscopy to the test object or to calibrate the examination. Alternatively, use may be made of further noninvasive measurement methods to provide further values that correlate with the substances to be examined in order, for example, to increase the accuracy (e.g., the reliability of the values) therewith or in order to exclude other conclusions or sources of errors during the analysis based on the measurements.

Alternatively or additionally, the arrangement may be configured such that, for the examination via the capture based on magnetic resonance spectroscopy, the arrangement is configured such that the arrangement is calibratable (e.g., in an automatic manner and with the aid of calibration substrates).

In one embodiment, the arrangement is configured as a portable arrangement with a reception region that is formed in a manner geared toward a body part (e.g., for receiving a finger). As a result, the measuring device may be transported, and hence, the measuring device may be used more flexibly.

In addition to the arrangement and the developments thereof, one or more of the present embodiments also relate to the method and the use of the arrangement (e.g., only the arrangement or in the form of the combinations of arrangement features that are specified in the developments) for noninvasively examining blood constituents that, for example, facilitate a miniaturization and a reliable (e.g., mobile) capture of blood substances.

### BRIEF DESCRIPTION OF THE DRAWINGS

FIG. 1 shows a magnetic resonance (MR) spectrum, as may appear in an exemplary embodiment;

FIG. 2 shows an MR spectroscopy arrangement for measuring blood sugar as a first mobile exemplary embodiment; and

FIG. 3 shows an MR spectroscopy arrangement for measuring blood sugar as an alternative second mobile exemplary embodiment.

### DETAILED DESCRIPTION

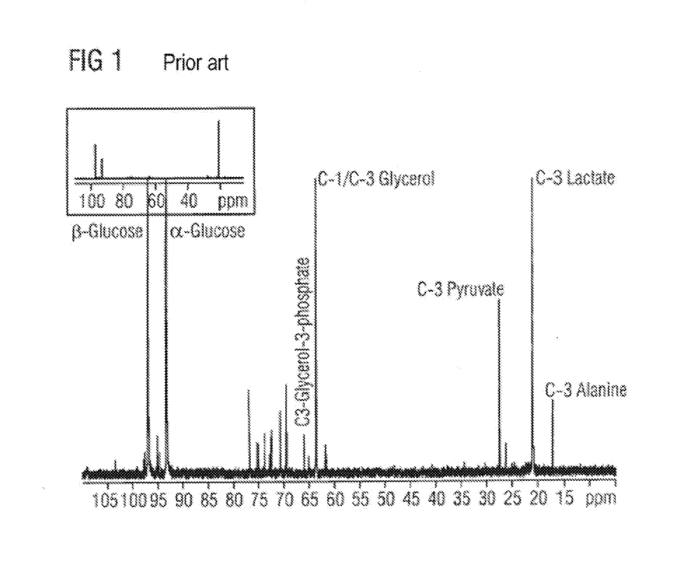

In FIG. 1, a spectrum that may appear when using an embodiment of an arrangement for noninvasively examining blood substances based on magnetic resonance spectroscopy (MRS) is shown.

According to one or more of the present embodiments, use is made of the fact that MRS may be used to identify and quantify various chemical substances in living tissue on account of a chemical shift.

The exemplary magnetic resonance spectroscopy (MR spectroscopy, MRS) measurement that is shown in FIG. 1 arises if, according to one or more of the present embodiments, the arrangement is configured such that a glucose concentration in the blood is measured using ¹H-MRS or ¹³C-MRS.

One or more of the present embodiments are distinguished from the idea disclosed in the literature (cf. Gruetter R., Novotny E. J., Boulware S. D., Rothman D. L., Mason G. F., Shulman G. I., Shulman R. G., Tamborlane W. V., “Direct measurement of brain glucose concentrations in humans by 13C NMR spectroscopy,” Proc Natl Acad Sci USA 1992, Dec. 15, 89(24), 12208) in that, therein, ¹³C-MRS only allows an insight into the cellular glucose metabolism, and this relates nonspecifically to the tissue of the human.

By contrast, in accordance with one or more of the present embodiments, human blood, which counts as connective and supporting tissue in the broadest sense, is used in the MRS magnetic resonance spectroscopy (e.g., ¹H-based or ¹³C-MRS-based) for noninvasively determining the glucose concentration. Further, one or more of the present embodiments are distinguished by virtue of concentrating on a small part of the body (e.g., on the finger) in accordance with the example (e.g., on account of the gearing toward the blood).

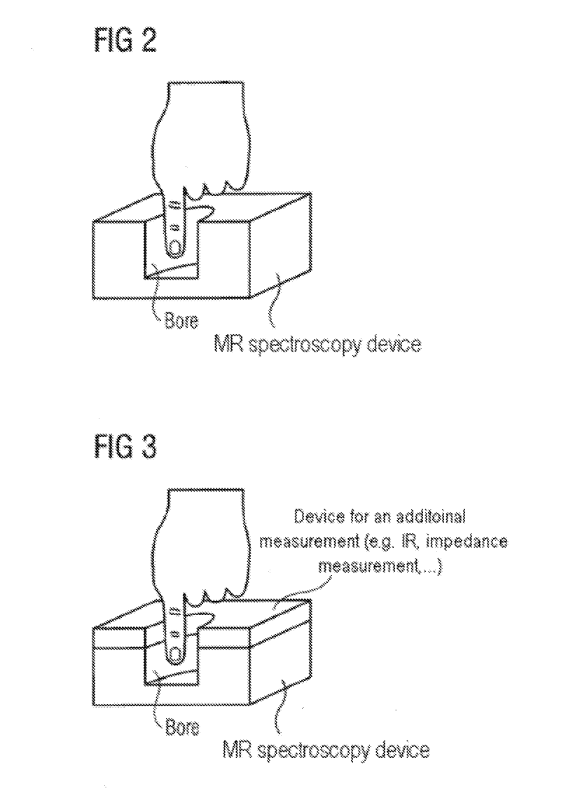

A first exemplary embodiment that is directed to examining the blood in the finger of a human is shown in FIG. 2.

The MRS device has a recess/bore for receiving the finger. Within the arrangement, the magnetic field that is provided for realizing the MRS may be produced using a permanent magnet or with the aid of other known technology (e.g., superconductor, electromagnet).

The homogeneity to be provided may be improved using specifically formed, switchable DC current coils (e.g., shim coils).

The field strength may vary between a few millitesla and several tesla. The device may be embodied in the form of a benchtop NMR spectrometer (cf. known device shapes such as, for example, “Spinsolve”, “picoSpin”, “NMReady”, “Pulsar”, “Fourier 60”), or the device may be larger and stand on the ground.

The presented system may be developed such that the system may be calibrated automatically or with the aid of calibration substrates, or the system may be combined with a conventional blood measurement as a standard. The measurement may be carried out using a finger, as presented, or using other suitable body parts.

By way of example, the measurement may be carried out on the wrist (e.g., by encircling the wrist) or by application on the skin surface by a probe at any point on the body that is suitable herefor.

The reliability of the values of the glucose concentration may be increased if the spectroscopy measurement is assisted by a preceding MRI image since the location of the spectroscopy measurement (e.g., blood vessels) may be precisely localized therewith. In accordance with a development, the blood flow may be localized in the arteries (e.g., using a Doppler measurement).

Information about the concentration of glucose in the blood may be provided by measuring glucose or by measuring other substances that correlate therewith (e.g., insulin receptor (INSR) or glucose transporters (e.g. “GLUT-1”)). In order to provide a higher accuracy and reliability, these measurements may also be combined with one another.

The MRS in accordance with the first exemplary embodiment and the mentioned variants and developments in this respect may be combined with other methods (e.g., infrared (IR) or microwave-based measurements) for obtaining a higher accuracy and reliability, as is shows in FIG. 3.

In addition, in the aforementioned examples, the MRS may also be combined with sensors (e.g., temperature sensors) in order to obtain a better measurement accuracy or in order to reduce the frequency of calibration required.

The presented arrangements and methods, or the use of MRS and the further measuring devices for determining the concentration of glucose in blood, may also be transferred, mutatis mutandis, to other substances in blood or in the body (e.g., alcohol, adrenaline, cortisol, or testosterone), and the presented arrangement and methods are not restricted to glucose examination only.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An arrangement for noninvasively examining at least parts of blood constituents,
wherein the arrangement is configured to examine the blood constituents based on at least magnetic resonance spectroscopy of a part of a human body.

2. The arrangement of claim 1, wherein the arrangement is configured for the examination by capture of at least one blood substance based on at least magnetic resonance spectroscopy.

3. The arrangement of claim 1, wherein the arrangement is configured for the examination by capture of at least the blood sugar concentration based on at least magnetic resonance spectroscopy.

4. The arrangement of claim 1, wherein the arrangement is configured for the examination by capture of at least one blood substance, at least via a marker substance that correlates with the blood substance, based on at least magnetic resonance spectroscopy.

5. The arrangement of claim 1, wherein the arrangement is configured for the examination by capture of the blood substance glucose via at least one marker substance that correlates with the blood substance glucose, based on at least magnetic resonance spectroscopy.

6. The arrangement of claim 1, wherein the arrangement is configured for the examination by capture of the blood sugar metabolism, via at least a correlating marker substance, based on at least magnetic resonance spectroscopy, and
wherein the arrangement is configured with means for evaluating the capture, the means for evaluating being configured such that the blood sugar concentration is derived from the spectrum that belongs to the marker substance.

7. The arrangement of claim 1, wherein, for the examination by capture based on magnetic resonance spectroscopy, the arrangement comprises a magnetic resonance imaging device, the magnetic resonance imaging device comprising:
means for carrying out a Doppler measurement;
means for carrying out an infrared measurement;
means for carrying out a microwave-based measurement
sensors, the magnetic resonance imaging device being configured in a manner functionally linked with the sensors such that the capture is carried out depending on a measurement that is effectuated by the sensors; or
any combination thereof.

8. The arrangement of claim 1, wherein, for the examination by the capture based on magnetic resonance spectroscopy, the arrangement is configured such that the arrangement is calibratable, in particular in an automatic manner and in particular with the aid of calibration substrates.

9. The arrangement of claim 1, wherein the arrangement is configured as a portable arrangement with a reception region that is formed in a manner geared toward the part of the human body.

10. The arrangement of claim 1, wherein the arrangement is configured for magnetic resonance spectroscopy such that a magnetic field is produced by a permanent magnet, a superconductor, and/or an electromagnet, or any combination thereof.

11. The arrangement of claim 1, wherein the arrangement has intermediate layers with an active or passive mode of action for the purposes of the magnetic resonance spectroscopy.

12. A method of using an arrangement for noninvasively examining at least parts of the blood constituents, the method comprising:
examining, by the arrangement, the blood constituents based on at least magnetic resonance spectroscopy of a part of a human body.

13. A method for noninvasively examining at least parts of blood constituents, the method comprising:
examining, by an arrangement, the blood constituents based on at least magnetic resonance spectroscopy of a part of a human body.

14. The method of claim 13, further comprising using a blood sample that was measured via an invasive method for calibration purposes,
wherein the examining comprises examining the blood constituents based on the calibration, the examining being carried out with the aid of noninvasive magnetic resonance spectroscopy.

15. The arrangement of claim 1, wherein the part of the human body comprises a human finger.

16. The arrangement of claim 5, wherein the blood substance glucose is a glucose transporter or an insulin receptor.

17. The arrangement of claim 6, wherein the arrangement is configured for the examination by capture of the blood sugar metabolism, based on a hydrogen atom nuclei, a carbon atom nuclei, or the hydrogen atom nuclei and the carbon atom nuclei.

18. The arrangement of claim 7, wherein the sensors comprise temperature sensors.

19. The arrangement of claim 8, wherein the arrangement is calibratable in an automatic manner and with the aid of calibration substrates.
